(19) United States
(12) Patent Application Publication
(10) Pub. No.: US 2007/0135869 A1
(43) Pub. Date: Jun. 14, 2007

(54) Apparatus for stimulating acupuncture points on a human face

(75) Inventor: Chiang-Chuan Chen, Taipei City (CN)

Correspondence Address: BIRCH STEWART KOLASCH & BIRCH, PO BOX 747, FALLS CHURCH, VA, 22040-0747, US

(73) Assignee: HEALTH & WELFARE TECHNOLOGY INC.

(21) Appl. No.: 11/399,535

(22) Filed: Apr. 7, 2006

(30) Foreign Application Priority Data
Dec. 13, 2005 (TW) 094143982

(51) Int. Cl. A61N 5/06

(52) U.S. Cl. 607/88

(57) ABSTRACT

An apparatus for stimulating acupuncture points on a human face comprises a mask and a plurality of light sources and control circuits. This apparatus projects optical energy onto the acupuncture points on the human face to stimulate them and promote health.

### BACKGROUND OF THE INVENTION

(a) Field of the Invention

The present invention relates to an apparatus for stimulating acupuncture points on a human face, more particularly to an apparatus for stimulating acupuncture points on a human face by optical energy.

(b) Description of the Prior Art

Acupuncture and moxibustion are holistically called acu-moxibustion for short (but the term “acupuncture” is used to refer to both acupuncture and moxibustion in the west.) Acupuncture is a method in which fine needles are inserted into a person's different acupuncture points (“acupoints” for short) at different depths, whereas moxibustion is a method in which different acupoints are smoldered by moxa. Acu-moxibustion gives people self-healing power, or can relieve pain or enhance a patient's resistance to diseases. According to conventional acu-moxibustion practices, filiform needles are applied on the acupoints located on the running course of meridians. When stimulation produces aching, numbness, and distension, this is called “arrival of qi” or “acuesthesia,” which can dredge qi and blood, regulate visceral functions, and treat diseases based on the arrival of qi. However, according to clinical practices, when needles are extracted after the treatment, the “arrival of qi” on the acupoints no longer exists, just like the expiration of drug potency. Therefore, patients are required to receive outpatient acupuncture treatment every other day to maintain therapeutic effects, thereby causing extreme inconvenience to the patients.

Moreover, as acupuncture is required to be administered by professional acupuncturists, patients should continue acupuncture treatment in the hospital, which not only causes inconvenience to the patients but is also a burden to the health care system. Furthermore, acupuncture is an interventional therapy, and if needles are administered wrongly, this easily causes damages to the human body. Therefore, conventional acu-moxibustion is time-consuming and easily causes the fear of repeated acupuncture treatment.

On the other hand, a lot of acupoints are clustered around the face. These acupoints are extremely helpful in promoting human health and enhancing human functions. Regarding major facial acupoints, for example, the stimulation of Tsuan Ochu acupoint can relieve eye fatigue, eliminate eye edema, and prevent wrinkles; the stimulation of Tayan acupoint can promote metabolism, can relieve eye fatigue and eliminate eye edema; the stimulation of Cheng Chi acupoint can enhance gastric functions and prevent the loosening of eye bags; the stimulation of Yi Ziang acupoint can relieve eye fatigue, eliminate eye edema, prevent skin loosening, relieve shoulder aching and nasal congestion, promote eye circulation and improve facial nerve palsy; the stimulation of Jia Che acupoint can effectively eliminate the fatness caused by the absorption of excessive saccharides; the stimulation of Di Cang acupoint can lower the temperature of the stomach and inhibit appetite; the stimulation of Ci Chiang acupoint can control hormonal secretion and treat stroke, facial nerve palsy, facial edema, tooth neuralgia, and neck aching; the stimulation of Chung Mei acupoint can refresh oneself, enhance vision, and improve eye hyperemia; the stimulation of Si Zhu Kong acupoint can eliminate facial edema and make the skin astringent; the stimulation of Tung Tzu Liao acupoint can eliminate edema; the stimulation of Si Bai acupoint can make the pupil clear and reduce the bloodshot condition; the stimulation of Lu Jiao acupoint can eliminate edema; the stimulation of Intang acupoint can relieve headache and dizziness and promote the health of nose; the stimulation of Xia Guan can eliminate wrinkles and relieve toothache; the stimulation of Chiuho acupoint can enhance the functions of small intestines; the stimulation of Yuyao acupoint can relieve eye fatigue and lessen ptosis (drooping eyelid); the stimulation of Guan Jiao acupoint can relieve eye fatigue and face neuralgia; the stimulation of Da Ying acupoint can eliminate edema.

Unfortunately, as one's face is related to one's appearance, conventional acupunctural practices may hurt the skin on the face. Consequently, patients are mostly unwilling to apply needles on their face. Moreover, some patients are afraid of needle application, and if acupuncturists apply the needles on the face, these patients can hardly accept this, thereby making the application of the acupoints on the face difficult.

### SUMMARY OF THE INVENTION

The primary object of the present invention is to provide an apparatus for stimulating acupoints on a human face. The present invention projects optical energy onto the acupoints on the face to stimulate them and promote human health.

To achieve the above-mentioned object, the present invention provides an apparatus for stimulating acupoints on a human face, comprising a mask and a plurality of light sources and control circuits, wherein the mask has an outer case and an inner case. The outer case is made of transparent materials whereas the inner case comprises a plurality of pores. The plurality of light sources is disposed inside the pores, such that when the light sources emit light, light rays can be projected onto a plurality of corresponding target regions. Then the control circuits are coupled to the corresponding light sources and output at least one control signal to the corresponding light sources, such that the light sources emit light at the target wavelength. When the light sources emit light, light rays enter the inner case by optical actions because the inner case is made of transparent materials. As light rays are propagated inside the inner case, they can be evenly projected onto the face by the reflection, diffusion, and transmission of light rays.

According to a preferred embodiment of the present invention, the target regions are the acupoints for Tsuan Ochu, Tayan, Cheng Chi, Ying Ziang, Jia Che, Di Cang, Ci Chiang, Chung Mei, Si Zhu Kong, Tung Tzu Liao, Si Bai, Lu Jiao, Intang, Xia Guan, Chiuho, Yuyao, Guan Jiao, and Da Ying.

According to a preferred embodiment of the present invention, the control signal is a pulse wave.

According to a preferred embodiment of the present invention, the frequency of the pulse wave lies between 140 Hz and 300 Hz.

According to a preferred embodiment of the present invention, the light sources are INFRARED, RED, Blue, or Green light.

According to a preferred embodiment of the present invention, the wavelength of the light sources lies between 360 nm and 1200 nm.

According to a preferred embodiment of the present invention, the control circuit outputs the control signal to the corresponding light source, such that the light source first emits light for 10 to 60 minutes and then stops emitting light. The duration of light emission can be adjusted based on needs.

According to a preferred embodiment of the present invention, the transparent materials are polycarbonate (PC.)

According to a preferred embodiment of the present invention, the mask comprises an anti-reflective layer, which is disposed between the is inner case and the outer case and reflects the light rays emitted from the light source onto the inner case.

According to a preferred embodiment of the present invention, the anti-reflective layer is made of Acrylonitrile-Butadiene-Styrene (ABS.)

According to a preferred embodiment of the present invention, the light source comprises a plurality of light-emitting diodes (LED.)

In summary, the present invention provides an apparatus for stimulating acupoints on a human face such that one's acupoints on the face can be stimulated without applying needles according to conventional acupunctural practices for health promotion. In this way, patients do not have to worry about the damages to the skin on the face caused by the stimulation of acupoints on the face and relieve the patients' fear of needle application. Moreover, they do not necessarily have to go to the hospital, but can have the stimulation of acupoints on the face at any time and place, thereby greatly lowering patients' burden. Therefore, the present invention can overcome the drawbacks caused by conventional acupunctural practices on the acupoints on the face, thereby facilitating the effective use of acupoints on the face.

To enable a further understanding of the objectives and the technological methods of the invention herein, the brief description of the drawings below is followed by the detailed description of the preferred embodiments.

### BRIEF DESCIPTION OF THE DRAWINGS

FIG. 1 shows a schematic view of an apparatus for stimulating acupoints on a human face according to a preferred embodiment of the present invention.

FIG. 2A shows a schematic view of a mask according to a preferred embodiment of the present invention.

FIG. 2B shows a schematic view of a mask according to a preferred embodiment of the present invention.

FIG. 2C shows a schematic view of a mask according to a preferred embodiment of the present invention.

FIG. 3 shows a circuit diagram depicting the stimulation of acupoints on a human face according to a preferred embodiment of the present invention.

FIG. 4 shows an acupoint diagram corresponding to the pores on an inner case according to a preferred embodiment of the present invention.

### DETAILED DESCRIPTION OF THE PREFERRED EMBODIMENTS

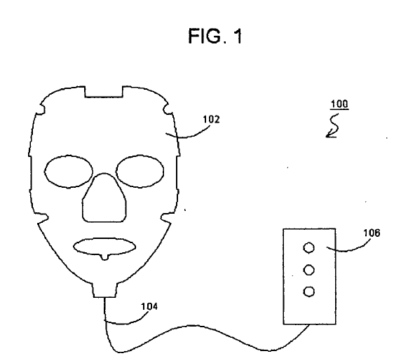

Referring to FIG. 1, an apparatus for stimulating acupoints on a human face 100 includes a mask 102, a signal cable 104, and a controller 106. The mask 102 is coupled to the signal cable 104, which is then coupled to the controller 106.

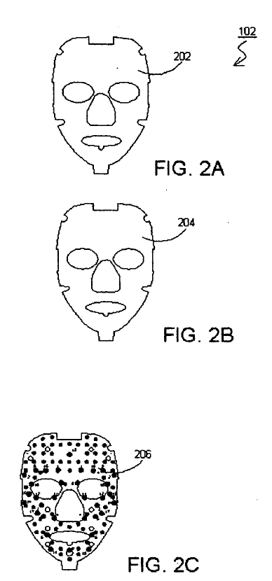

Referring to FIG. 2A, 2B, and 2C, the mask 102 includes an outer case 202, an anti-reflective layer 204, and an inner case 206, wherein the outer case 202 intercalates or binds the anti-reflective layer 204 and the inner case 206, such that the outer case 202, the anti-reflective layer 204, and the inner case 206 are combined to form the mask 102, wherein the anti-reflective layer 204 is made of Acrylonitrile-Butadiene-Styrene (ABS.) The anti-reflective layer 204 is disposed between the inner case 206 and the outer case 202 and can project the light source onto the inner case 206. Moreover, the inner case is made of polycarbonate (PC) and comprises a plurality of pores depicted as dots according to FIG. 2C. The plurality of light sources is LEDs (not shown in the drawing), which are coupled to the signal cable 104. These light sources are disposed inside the pores such that when these light sources emit light, the light rays are projected onto the plurality of corresponding target regions.

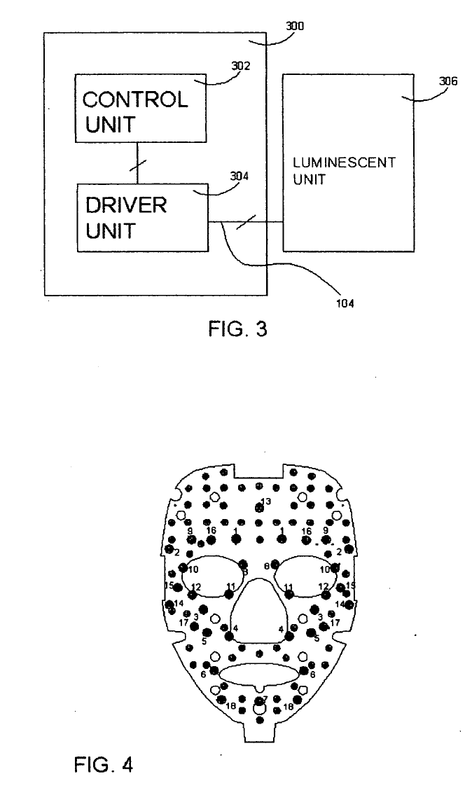

Referring to FIG. 3, a control circuit 300 comprises a control unit 302 and a drive unit 304. The control circuit 300 is disposed inside the controller 106. A luminescent unit 306, which is formed by a plurality of LEDs, is disposed inside the pores on the inner case. The control unit 302 can be taken as a microprocessor or a pulse wave generator to output at least one pulse signal. A pulse signal input drive unit is used to supply power to the light source (This part is deemed conventional circuit technology so that it will no longer be mentioned here.)

When the user is using the apparatus for stimulating acupoints on a human face 100, the mask 102 is first placed on the face such that the mask 102 covers the hollows on one's eyes, nose, and mouth, and is flatly attached onto the surface of the face. When the user turns on the power of the controller 106, the light source starts to emit light. Then the light rays emitted by the light source are projected onto the target region. As the inner case 206 is made of transparent materials, light rays enter the inner case 206 by optical actions. Due to the transmission of light rays inside the inner case 206, light rays can be emitted from the inner case 206 by light reflection, refraction, and scattering. Then the light rays emitted from the inner case 206 are reflected to the inner case 206 via the anti-reflective layer 204, such that optical energy is not lost, but is collected in the inner case 206 and projected onto the face. Therefore, by means of the above actions, light rays can be evenly projected onto the target region on the face.

The control signal is a pulse wave with frequency between 140 Hz and 300 Hz. The light source is INFRARED, RED, BLUE, or GREEN light with wavelength between 360 nm and 1200 nm. The control circuit 302 outputs control signals to the corresponding light source via the drive unit 304 such that the light source emits light for 10 to 60 minutes and then stops emitting light in order to prevent the light from hurting the skin. The light source is formed by a plurality of LEDs (not shown in the drawing.)

Referring to FIG. 4, the dots depict the location of the pores on the face. Dot 1 denotes the Tsuan Ochu acupoint; dot 2 denotes the Tayan acupoint; dot 3 denotes the Cheng Chi acupoint; dot 4 denotes the Ying Ziang acupoint; dot 5 denotes the Jia Che acupoint; dot 6 denotes the Di Cang acupoint; dot 7 denotes the Ci Chiang acupoint; dot 8 denotes the Chung Mei acupoint; dot 9 denotes the Si Zhu Kong acupoint; dot 10 denotes the Tung Tzu Liao acupoint; dot 11 denotes the Si Bai acupoint; dot 12 denotes the Lu Jiao acupoint; dot 13 denotes the Intang acupoint; dot 14 denotes the Xia Guan acupoint; dot 15 denotes the Chiuho acupoint; dot 16 denotes the Yuyao acupoint; dot 17 denotes the Guan Jiao acupoint; dot 18 denotes the Da Ying acupoint. Other dots also denote pores on the face, and LEDs can also be disposed in the pores. Referring to the above-mentioned, each of the pores on the inner case 206 corresponds to a specific acupoint on the face. When the user uses the apparatus for stimulating acupoints on the human face 100, the corresponding acupoints (that is the target regions) on the face will absorb more optical energy, which can stimulate the acupoints on the face for health promotion.

The dots disclosed in FIG. 4 are only cited as examples. However, for those who are skilled in the prior art, the location of the target region for optical energy projection can change with reference to different designs, so that those who are familiar with the prior art can adjust their embodiment accordingly.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. An apparatus for stimulating acupoints on a human face, comprising:
a mask, having an outer case and an inner case, wherein said outer case is made of transparent materials and said inner case has a plurality of pores;
a plurality of light sources, disposed inside said pores, such that when said light sources emit light, light rays are projected onto a plurality of corresponding target regions; and
a control circuit, coupled to said light sources, outputting at least one control signal to said corresponding light sources, such that said light sources emit light at a target wavelength;
during the light emission of said light sources, light rays enter said inner case by optical actions because said inner case is made of transparent materials, thereby making light rays being evenly projected onto the face due to the transmission of light rays inside said inner case.

2. The apparatus for stimulating acupoints on a human face as claimed in claim 1, wherein said target region corresponds to the Tsuan Ochu acupoint.

3. The apparatus for stimulating acupoints on a human face as claimed in claim 1, wherein said target region corresponds to the Tayan acupoint.

4. The apparatus for stimulating acupoints on a human face as claimed in claim 1, wherein said target region corresponds to the Cheng Chi acupoint.

5. The apparatus for stimulating acupoints on a human face as claimed in claim 1, wherein said target region corresponds to the Ying Ziang acupoint.

6. The apparatus for stimulating acupoints on a human face as claimed in claim 1, wherein said target region corresponds to the Jia Che acupoint.

7. The apparatus for stimulating acupoints on a human face as claimed in claim 1, wherein said target region corresponds to the Di Cang acupoint.

8. The apparatus for stimulating acupoints on a human face as claimed in claim 1, wherein said target region corresponds to the Ci Chiang acupoint.

9. The apparatus for stimulating acupoints on a human face as claimed in claim 1, wherein said target region corresponds to the Chung Mei acupoint.

10. The apparatus for stimulating acupoints on a human face as claimed in claim 1, wherein said target region corresponds to the Si Zhu Kong acupoint.

11. The apparatus for stimulating acupoints on a human face as claimed in claim 1, wherein said target region corresponds to the Tung Tzu Liao acupoint.

12. The apparatus for stimulating acupoints on a human face as claimed in claim 1, wherein said target region corresponds to the Si Bai acupoint.

13. The apparatus for stimulating acupoints on a human face as claimed in claim 1, wherein said target region corresponds to the Lu Jiao acupoint.

14. The apparatus for stimulating acupoints on a human face as claimed in claim 1, wherein said target region corresponds to the Intang acupoint.

15. The apparatus for stimulating acupoints on a human face as claimed in claim 1, wherein said target region corresponds to the Xia Guan acupoint.

16. The apparatus for stimulating acupoints on a human face as claimed in claim 1, wherein said target region corresponds to the Chiuho acupoint.

17. The apparatus for stimulating acupoints on a human face as claimed in claim 1, wherein said target region corresponds to the Yuyao acupoint.

18. The apparatus for stimulating acupoints on a human face as claimed in claim 1, wherein said target region corresponds to the Guan Jiao acupoint.

19. The apparatus for stimulating acupoints on a human face as claimed in claim 1, wherein said target region corresponds to the Da Ying acupoint.

20. The apparatus for stimulating acupoints on a human face as claimed in claim 1, wherein said control signal is a pulse wave.

21. The apparatus for stimulating acupoints on a human face as claimed in claim 20, wherein the frequency of said pulse wave lies between 140 Hz and 300 Hz.

22. The apparatus for stimulating acupoints on a human face as claimed in claim 1, wherein said control circuit outputs said control signal to a corresponding light source, such that said light source emits light for 10 to 60 minutes and then stops emitting light.

23. The apparatus for stimulating acupoints on a human face as claimed in claim 1, wherein said transparent material is polycarbonate.

24. The apparatus for stimulating acupoints on a human face as claimed in claim 1, wherein said mask comprises an anti-reflective layer disposed between said inner case and said outer case, such that light rays emitted from said light source are reflected to said inner case.

25. The apparatus for stimulating acupoints on a human face as claimed in claim 1, wherein said anti-reflective layer is made of Acrylonitrile-Butadiene-Styrene.

26. The apparatus for stimulating acupoints on a human face as claimed in claim 1, wherein said light source is formed by a plurality of light-emitting diodes (LED.)

27. The apparatus for stimulating acupoints on a human face as claimed in claim 1, wherein the wavelength of said light source is between 360 nm and 1200 nm.

28. The apparatus for stimulating acupoints on a human face as claimed in claim 1, wherein said light source is RED light.

29. The apparatus for stimulating acupoints on a human face as claimed in claim 1, wherein said light source is BLUE light.

30. The apparatus for stimulating acupoints on a human face as claimed in claim 1, wherein said light source is GREEN light.

31. The apparatus for stimulating acupoints on a human face as claimed in claim 1, wherein said light source is INFRARED light.
